(19) United States
(12) Patent Application Publication
(10) Pub. No.: US 2022/0110384 A1
(43) Pub. Date: Apr. 14, 2022

(54) PROTECTIVE TEMPERATURE-SENSING FACE SHIELD WITH TEMPERATURE DISPLAY

(71) Applicant: Antionette Olivarez, Porter Ranch, CA (US)

(72) Inventor: Antionette Olivarez, Porter Ranch, CA (US)

(21) Appl. No.: 17/163,830

(22) Filed: Feb. 1, 2021

Related U.S. Application Data
(60) Provisional application No. 63/091,654, filed on Oct. 14, 2020.

(51) Int. Cl. A41D 13/11; G01K 13/20; G01K 1/02

(52) U.S. Cl. CPC A41D 13/1161; A41D 2300/33; G01K 1/02; G01K 13/223

(57) ABSTRACT

A temperature-sensing face covering, such as a face shield, with a temperature sensor and display unit therein to provide for hands free temperature readings that go wherever the individual goes and continue to measure the individual's temperature throughout the day. The temperature-sensing face covering is simple and does not require the sites of work or other places to have any additional equipment, and provides continual readings that could very easily change throughout the day and be missed because at the beginning of the day the individual did not have a fever.

### CROSS-REFERENCE TO RELATED APPLICATION

The present application is related to and claims priority from prior provisional application Ser. No. 63/091,654, filed Oct. 14, 2020, the entire application of which is incorporated herein by reference.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

### 1. Field of the Invention

The present invention relates generally to the field of face coverings and more specifically relates to a temperature-sensing face shield or facemask.

### 2. Description of the Related Art

Facemasks and face shields are common items worn worldwide during flu season and pandemics. Wearing a facemask or a face shield helps prevent the wearer from spreading any infectious agent(s) he or she may currently have, including bacteria and viruses. They may also provide some level of protection against coming into contact with an infectious agent shed by another person.

The guidelines issued by the United States Food and Drug Administration (FDA) include using temperature measurements as part of an assessment to determine if a person has an illness that commonly causes elevated body temperature, such as COVID-19. The United States Center for Disease Control and Prevention (CDC) recommends using indications of a fever equal to or higher than 100.4° F. as a guide to check for possible infection.

Currently, many places are requiring everyone who enters to stop at the entrance and have their temperature taken. This requires many places of business to set up a temperature-check local and system, and buy temperature-sensing equipment. However, an individual could be afebrile when they enter the building, but develop a fever over the course of the day and not know he or she was febrile because he or she was afebrile when checked earlier in the day. Alternately, people may be walking around in areas where such temperature checks are not performed and never realize they are ill. Or they walk about not knowing that they are ill. An easy way for individuals to monitor their temperature on the go throughout the day would provide an early warning indicator of illness.

Various attempts have been made to solve the problems that may be found in the related art but have thus far been unsuccessful. A need exists for a reliable temperature-sensing face covering to avoid the above-mentioned problems.

### BRIEF SUMMARY OF THE INVENTION

In view of the foregoing disadvantages inherent in the known art, the present invention provides a novel temperature-sensing face covering. The general purpose of the present invention, which will be described subsequently in greater detail, is to, provide a temperature sensor on a facemask or face shield. Such temperature-sensing facemasks and face shields provide for hands free temperature readings that go wherever the individual goes, and continue to measure the individual's temperature throughout the day. The temperature-sensing face covering is simple and does not require the sites of work or other places to have any additional equipment, and provides continual readings that could very easily change throughout the day and be missed because at the beginning of the day the individual did not have a fever.

Such masks are also useful in the healthcare fields. Individuals who work in healthcare frequently are exposed to viruses and other droplet or airborne illnesses. The ability for the individual to wear their thermometer provides for rapid assessment and intervention, both in routine healthcare situations and crisis pandemic situations.

The features of the invention that are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures that accompany the written portion of this specification illustrate embodiments and method(s) of use for the present invention, a temperature-sensing face covering, constructed and operative according to the teachings of the present invention.

FIG. 1 shows a perspective view illustrating a temperature-sensing face shield according to an embodiment of the present invention.

FIG. 2 shows a perspective view illustrating a temperature-sensing face shield according to an embodiment of the present invention.

The various embodiments of the present invention will hereinafter be described in conjunction with the appended drawings.

### DETAILED DESCRIPTION

As discussed above, embodiments of the present invention relate to a temperature-sensing face covering and more particularly to a face shield with a temperature sensor.

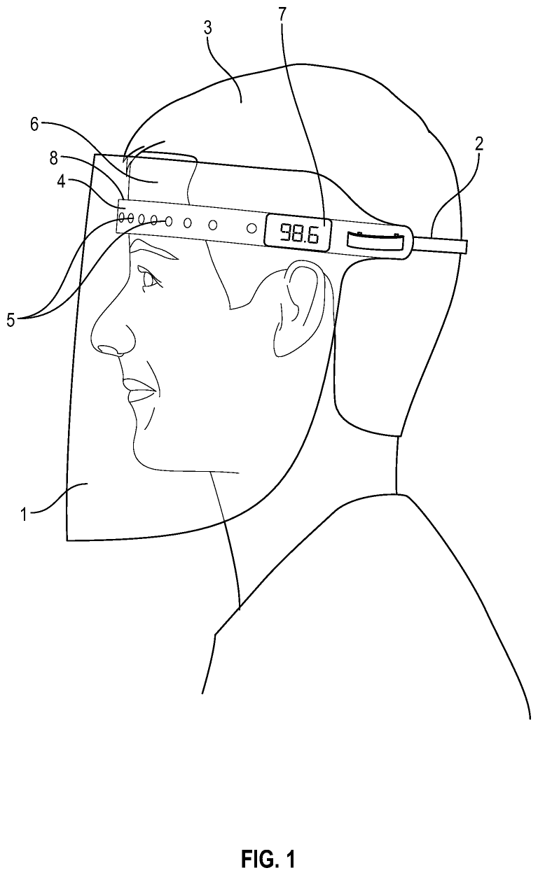

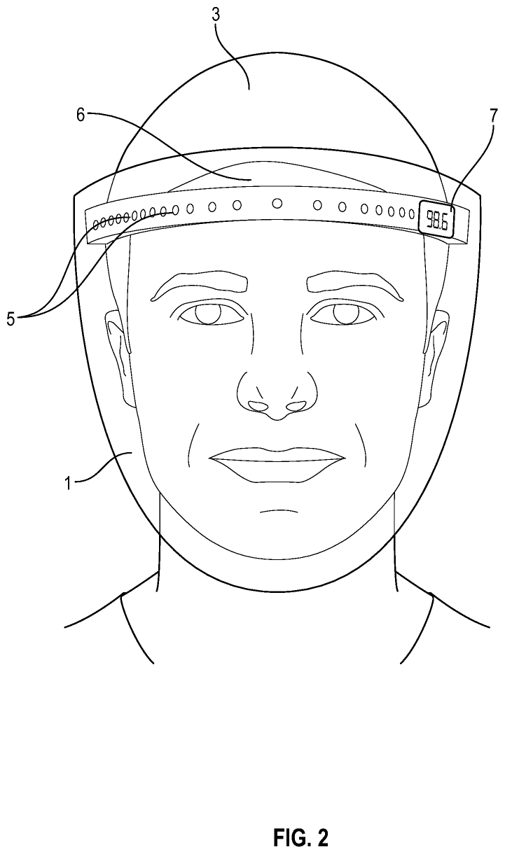

Referring now to the drawings FIGS. 1-2, the temperature-sensing face shield has a clear shield part 1 and a headband part 2 that holds the clear shield part 1 in front of a wearer's face 3. The headband part 2 may include padding 4 around the section that comes in contact with the wearer's face 3 to increase comfort when worn for extended durations of time. The temperature-sensing face shield contains one or more temperature sensors 5 in the headband 2, preferably located so that the temperature sensor(s) 5 will be held across the wearer's forehead 6.

The temperature-sensing face shield also includes a temperature emblem, marker, or other display 7, which is operably connected to the temperature sensor(s) 5 so as to display the current temperature reading. The display 7 may be located anywhere on the shield part 1 or headband part 2 as long as the display 7 does not obstruct the wearer's vision. Alternately, the emblem, marker, or other display 6 may display a color if the wearer's temperature is below 100.4° F. and change colors to display a different color if the wearer's temperature is over 100.4° F. The emblem, marker, or other display 6 may also both display the current temperature reading from the temperature sensor 5 and a color indication of whether or not the current temperature reading is below or above 100.4° F. The emblem, marker, or other display 6 may be located on either side of the mask portion 1, and may come in a variety of sizes and colors.

The temperature sensor(s) 5 may be infrared temperature sensors. The headband may include a small lip 8 that separates the infrared temperature sensor(s) 5 from the wearer's skin 3, allowing the infrared temperature sensor(s) 5 to continually read the wearer's temperature.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claim:

1. A temperature-sensing face covering with display comprising:
a. a clear face covering portion having an inside side and an outside side;
b. a display operably connected to the outside side of the face covering portion so as to be visible when a user is wearing the face covering;
c. a headband part operably connected to the face covering portion so as to hold the face covering portion in front of the user's face when worn; and
d. one or more temperature sensors,
wherein the one or more temperature sensors are operably connected to or embedded within the headband part,
wherein the one or more temperature sensors are operably connected to the display in order to provide a body temperature indication for a wearer.

2. The temperature-sensing face covering with display according to claim 1, wherein the one or more temperature sensors are operably connected to or embedded with a section of the headband part that comes into contact with the user's forehead when worn.

3. The temperature-sensing face covering with a display according to claim 1, wherein the one or more temperature sensors are infrared temperature sensors.

4. The temperature-sensing face covering with a display according to claim 1, wherein the display is a programmable digital display, and wherein the display indicates the user's body temperature in numerical form.

5. The temperature-sensing face covering with a display according to claim 1, wherein the display is programmable digital display, and wherein the display shows a first color when the user's body temperature is 100.4° F. or lower, and a second color when the user's body temperature is higher than 100.4° F.

6. The temperature-sensing face covering with a display according to claim 3, wherein the headband part further comprises:
e. a headband lip, wherein the one or more temperature sensors are operably positioned on the headband part so the headband lip creates a gap between the one or more temperature sensors and the user's skin.

7. The temperature-sensing face covering with a display according to claim 3, wherein the headband part further comprises:
e. a headband padding part, wherein the headband padding part is a soft, padded material that forms or covers the section of the headband part that comes into contact with the user's forehead when worn.

8. A temperature-sensing face covering with display comprising:
a. a clear face covering portion having an inside side and an outside side;
b. a display operably connected to the outside side of the face covering portion so as to be visible when a user is wearing the face covering;
c. a headband part operably connected to the face covering portion so as to hold the face covering portion in front of the user's face when worn,
d. one ore more temperature sensors,
wherein the one or more temperature sensors are operably connected to or embedded with a section of the headband part that comes into contact with the user's forehead when worn, and
wherein the one or more temperature sensors are operably connected to the display in order to provide a body temperature indication for a wearer; and
e. a headband padding part, wherein the headband padding part is a soft, padded material that forms or covers the section of the headband part that comes into contact with the user's forehead when worn,
wherein the display is programmable digital display that indicates the user's body temperature in numerical form, and wherein the display shows a first color when the user's body temperature is 100.4° F. or lower, and a second color when the user's body temperature is higher than 100.4° F.

9. The temperature-sensing face covering with a display according to claim 8, wherein the headband part further comprises:
f. a headband lip, wherein the one or more temperature sensors are operably positioned on the headband part so the headband lip creates a gap between the one or more temperature sensors and the user's skin, and
wherein the one or more temperature sensors are infrared temperature sensors.
